(19) United States
(12) Patent Application Publication
(10) Pub. No.: US 2019/0130511 A1
(43) Pub. Date: May 2, 2019

(54) SYSTEMS AND METHODS FOR INTERACTIVE DYNAMIC LEARNING DIAGNOSTICS AND FEEDBACK

(71) Applicant: ACT, Inc., Iowa City, IA (US)

(72) Inventors: ALINA VON DAVIER, Iowa City, IA (US); STEPHEN POLYAK, Iowa City, IA (US); KURT PETERSCHMIDT, Iowa City, IA (US); PRAVIN CHOPADE, Iowa City, IA (US); MICHAEL YUDELSON, Iowa City, IA (US); JIMMY DE LA TORRE, Iowa City, IA (US); PAMELA PAEK, Iowa City, IA (US)

(21) Appl. No.: 15/802,404

(22) Filed: Nov. 2, 2017

(51) Int. Cl. G06Q 50/20; G09B 7/02; G09B 7/06; A61B 5/16; G06F 3/0481

(52) U.S. Cl. CPC G06Q 50/205; G09B 7/02; G06F 3/0481; A61B 5/167; G09B 7/06

(57) ABSTRACT

Systems and methods for dynamically assessing and providing feedback to a learner include displaying a set of assessment questions on a graphical user interface, obtaining a set of responses corresponding to the assessment questions, obtaining a set of diagnostic scoring rules including a set of diagnostic parameters corresponding to each assessment question and a response key, obtaining a set of learner-specific behavioral parameters, applying the set of diagnostic scoring rules to the set of responses to generate a learner response matrix, generating a learner attribute profile by applying as a set of probabilities of mastering each learning category to the learner response matrix, and estimating a learner response to a subsequent assessment question by applying a cognitive diagnostic model (CDM) or a Bayesian knowledge tracing (BKT) process to the learner attribute profile to the learner attribute profile.

### TECHNICAL FIELD

The disclosed technology relates generally to digital testing, and more particularly various embodiments relate to systems and methods for dynamic learning diagnostics and feedback using multi-dimensional data.

### BACKGROUND

Digital testing has become more prevalent, enabling the collection of digital testing data and led to the development of analytical tools and models for interactively assessing the knowledge and providing feedback to test takers to improve the learning experience. Some of these tools are based on the-so-called cognitive diagnostic model (CDM) in which test-takers are assessed according to their performance on a predefined set of knowledge quanta often referred to as skills or attributes. The test-taker's performance with respect to each attribute on one examination may be expressed in terms of a probability that the learner has mastered the respective skill i.e. CDMs provide a probability of mastering each attribute. Test-takers receive attribute-specific feedback based on a one-time test (summative) based on psychometrics theory (Item Response Theory). The CDM may include a mapping of the questions on the test to a set of attributes, sometimes referred to as a Q-matrix. The Q-matrix expresses whether a particular skill is required for each assessment item. The CDMs are mostly used for cross-sectional testing data.

In addition to the CDM, other models have been applied to capture learning trajectories: how does a learner master an attribute over time, while accounting for the number of practice exercises and for the feedback and assistance that the learner received. One such model is the Bayesian Knowledge Tracing (BKT). BKT also assesses skills, just like the CDM, but BKT accounts for the learning progress over time. For example, the BKT works by estimating the probability that a learner masters a specific skill across multiple attempts by the learner to apply the skill. The BKT uses longitudinal data. As compared to BKT, CDM provides a finer grained learner skills diagnostic assessment.

In addition to the CDM and other learning models, additional research has been done on the comparison of learners' cognitive abilities and performance to their socioemotional and behavioral characteristics. These characteristics have been shown to affect the efficiency and speed at which each learner masters a skill. In addition to collecting the data from the digital system with which the learner interacts, data may also be acquired using additional tests measuring socioemotional parameters. Existing analytical instruments assess learner's capabilities using behavioral analytics (a hybrid of machine learning and CDM), and may provide useful feedback to the learner. The feedback may include, for example, tips on time management, provide star ratings per area along with an indication of what people with 3 stars typically exhibit, recommend resources for learners to review to improve their behavioral ratings or other behaviors that have been shown to affect learning performance. However, existing approaches do not present an integrated solution to assess, diagnose, and provide feedback to a learner dynamically based on CDM and, socioemotional, and behavioral analytics.

### BRIEF SUMMARY OF EMBODIMENTS

Systems and methods for interactive dynamic learning diagnostics and feedback using multi-dimensional data are disclosed. Some embodiments of the disclosure provide a method for dynamic learning diagnostics and feedback that includes integrated analysis of learner's performance with respect to mastering one or more granular target skills. The integrated analysis may include applying a CDM model and/or a BKT model to learner responses to one or more assessment questions. Some embodiments of the disclosure also include using one or more socioemotional and/or behavioral characteristics of the learner to alter the CDM and/or BKT model performance. The output of the integrated analysis may be in the form of a learner profile indicating the probabilities that the learner has mastered or still needs to improve upon a set of skills. Embodiments of the disclosure may also use the behavioral and socioemotional data on the learner to provide feedback, insight, and recommendations to improve studying behaviors and strategies and achieve one or more educational goals set by the learner, the system, or another user.

Embodiments of the disclosure also provide a user interface, integrated with a dynamic learning diagnostics and feedback system. This interface may be used to present the learner with a set of resources selected to assist him or her in achieving his or her educational goal. The learner interface may include a graphical user interface, for example, presented on a mobile device, which may be configured to obtain responses to a set of assessment questions, obtain socioemotional and behavioral data, present an analytical report of the learner's performance with respect to the learner's goals and one or more skills, and deliver feedback to the learner to improve.

Other features and aspects of the disclosed technology will become apparent from the following detailed description, taken in conjunction with the accompanying drawings, that illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1A illustrates an example system for dynamic learning diagnostics and feedback, consistent with embodiments disclosed herein.

FIG. 1B illustrates an example multidimensional holistic model for dynamic learning diagnostics and feedback, consistent with embodiments disclosed herein.

FIG. 1C illustrates a schematic diagram of an example interactive learner interface, consistent with embodiments disclosed herein.

FIG. 2 is a flow chart illustrating an example method for providing dynamic learning diagnostics and feedback, consistent with embodiments disclosed herein.

FIG. 3 is a flow chart illustrating an example Bayesian knowledge tracing process, consistent with embodiments disclosed herein.

FIG. 4 is a flow chart illustrating an example learner analytics process, consistent with embodiments disclosed herein.

FIG. 5 illustrates an example Q-matrix as used in connection with embodiments disclosed herein.

FIG. 6 is an example representation of an output from a CDM analysis as used in connection with embodiments disclosed herein.

FIG. 7 is a diagram illustrating a Bayesian knowledge tracing process as used in connection with embodiments disclosed herein.

FIG. 8 is a diagram illustrating a Bayesian knowledge tracing process represented as a hidden Markov model, consistent with embodiments disclosed herein.

FIG. 9 illustrates an example computing system that may be used in implementing various features of embodiments of the disclosed technology.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

Embodiments of the technology disclosed herein are directed toward systems and methods for interactive dynamic learning diagnostics and feedback. More specifically, examples of the disclosed technology apply a CDM assessment diagnostic process together with a BKT process and behavioral and socioemotional analytics to dynamically identify and trace learners' strengths and weaknesses and provide adaptive feedback.

In some embodiments, a computer implemented method of dynamically assessing and providing feedback to a learner includes displaying a set of assessment questions on a learner interface and obtaining a set of responses corresponding to the assessment questions from the learner interface. For example, the learner interface may be a graphical user interface. The method may also include obtaining a set of diagnostic scoring rules, wherein each scoring rule includes a set of diagnostic parameters corresponding to each assessment question and a response key. The method may also include obtaining a set of learner-specific behavioral parameters, applying the set of diagnostic scoring rules to the set of responses to generate a learner response matrix, generating a learner attribute profile by applying as a set of probabilities of mastering each learning category to the learner response matrix, and estimating a learner response to a subsequent assessment question by applying a CDM or a BKT process to the learner attribute profile. For example, learning categories may include subjects, such as math, science, reading, history, or other subjects as known in the art.

In some examples, the learner attribute profile includes the set of learner-specific behavioral parameters. The learner response matrix may include a list of categories and a level of skill accrued by the learner with respect to each category. Examples of the BKT process may include displaying, with the learner interface, a subset of assessment questions wherein each question in the subset is selected from a common category, determining a skill-specific mastery value and updated learner attribute profile by tracing an accuracy of each sequential response to each question of the subset of assessment questions, and predicting a learner response to a subsequent assessment question from the subset of assessment questions as a function of the skill-specific mastery value and the updated learner attribute profile.

In some embodiments, the BKT process also includes generating a multi-state Bayesian knowledge vector corresponding to the common category. For example, the multi-state Bayesian knowledge vector may include a first parameter indicating whether the category is presently mastered and a second parameter indicating the probability that the category will be mastered within a threshold timeframe as a function the skill-specific mastery value and updated learner attribute profile.

Some embodiments of the method also include determining if any of the learner-specific behavioral parameters correlate to at-risk behavior. The method may also include correlating the set of learner-specific behavioral parameters to the learning rate and the learner attribute profile. In some examples, the method includes presenting, a set of behavioral improvement recommendations to correct at-risk behavior, presenting a set of behavioral improvement recommendations to increase the skill-specific mastery value, or presenting a set of behavioral improvement recommendations to increase a learning rate to the learner interface.

In some examples, the learner-specific behavioral parameters include a type of learning resource accessed by the learner, a time spent by the learner on a task, a participation level of the learner with an interactive interface, or a persistence ratio of a number of times retaking an assessment compared with the probability that one or more skills from the category will be mastered. Example of obtaining the learner-specific behavioral parameters may include receiving behavioral indications from a learner input device. For example, the learner input device may include a mouse, a microphone, a keyboard, or a touchscreen. Determining if any of the learner-specific behavioral parameters correlate to at-risk behavior may include obtaining historical behavioral data from a historical assessment database.

Some embodiments disclosed herein provide a system for dynamically assessing and providing feedback to a learner. The system may include a learner interface, a data store, and an assessment analytics logical circuit. For example, the assessment analytics logical circuit may include a processor and a non-transitory medium with computer executable instructions embedded thereon. The computer executable instructions may cause the processor to display a set of assessment questions on the learner interface, obtain, from the learner interface, a set of responses corresponding to the assessment questions, obtain a set of diagnostic scoring rules, each scoring rule comprising a set of diagnostic parameters corresponding to each assessment question and a response key, and obtain a set of learner-specific behavioral parameters. In some examples, the assessment analytics logical circuit may apply the set of diagnostic scoring rules to the set of responses to generate a learner response matrix, generate a learner attribute profile by applying as a set of probabilities of mastering each learning category to the learner response matrix, and estimate a learner response to a subsequent assessment question by applying a CDM or a Bayesian knowledge tracing (BKT) process to the learner attribute profile.

Some examples of the assessment analytics logical circuit may display a subset of assessment questions on the learner interface, wherein each question in the subset is selected from a common category, determine a skill-specific mastery value and updated learner attribute profile by tracing an accuracy of each sequential response to each question of the subset of assessment questions, and predict a learner response to a subsequent assessment question from the subset of assessment questions as a function of the skill-specific mastery value and the updated learner attribute profile. The system may also generate a multi-state Bayesian knowledge vector corresponding to the common category, the multi-state Bayesian knowledge vector comprising a first parameter indicating whether the category is presently mastered and a second parameter indicating the probability that the category will be mastered within a threshold timeframe as a function the skill-specific mastery value and updated learner attribute profile.

In some examples, the assessment analytics logical circuit determine if any of the learner-specific behavioral parameters correlate to at-risk behavior. The system may also correlate the set of learner-specific behavioral parameters to the learning rate and the learner attribute profile. In some embodiments, the system may present a set of behavioral improvement recommendations on the learner interface. In some examples, the system receives behavioral indications from a learner input device. For example, the learner input device may include a mouse, a microphone, a keyboard, or a touchscreen. The system may also determine if any of the learner-specific behavioral parameters correlate to at-risk behavior by obtaining historical behavioral data from a historical assessment database.

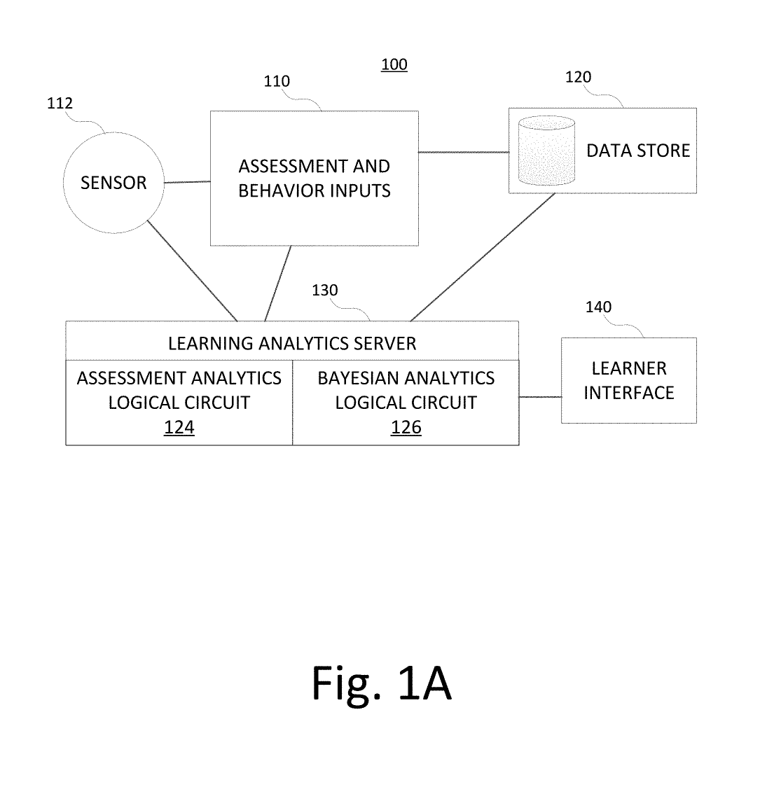

FIG. 1A illustrates an example system for dynamic learning diagnostics and feedback. In some embodiments, a system for dynamic learning diagnostics and feedback 100 includes assessment and behavior inputs logical circuit 110. For example, assessment and behavior inputs 110 may include inputs from an electronic or paper assessment or examination received from a scanner, a computer, a tablet, a mobile device, or other electronic input devices as known in the art. Assessment and behavior inputs logical circuit 110 may also include a graphical user interface, for example, to present an electronic examination to a user and accept responses to the examination. The responses may be in the form of multiple choice, short answer, essay, or other response formats as would be known in the art. In some embodiments, assessment and behavior inputs logical circuit 110 may be incorporated in learner interface 140.

Behavioral inputs may include data from a one or more examination preparation tools. The data may include study parameters such as the total amount of study time, the amount of study time per day, the total and average amounts of uninterrupted study periods. The data may be specific to or categorized by skill category or attribute. In some examples, the data includes responsive behavior, such as number of attempts, success and failures. For example, if a learner incorrectly answers one or more practice questions, does the learner immediately take corrective action, e.g., by studying the particular skill area and/or continuing to practice, or does the learner give up.

Assessment and behavior inputs may be received from assessment and behavior logical circuit 110. Such inputs may further include socioemotional inputs. For example, the learner may be given personality assessment tests to correlate a learning or behavior pattern to a particular set of personality traits to adaptively prepare a targeted learning feedback report. The socioemotional inputs may also include information from a learner's social media feeds and/or web browsing history, e.g., to determine how a learner spends his or her free time, if the learner interactively participates in educational or non-educational social media conversations, and how those patterns correlate over a particular population to successful learning behaviors.

In some examples, data from a sensor 112 may also be included in the assessment and behavioral inputs 110. For example, sensor 112 may include a camera configured to monitor a learner's concentration, eye movements, posture, or other characteristics visibly demonstrable while the learner studies, practices, takes assessments, or otherwise interacts with the system. Sensor 112 may also include an interactive device such as a mobile app, answer clicker, or other device that enables interaction with the system. Sensor 112 may also include a biosensor configured to monitor biodata, such as a learner's vital signs, brain activity (e.g., via an EKG or MEG), or other biodata that may indicate a learner's physiological state during assessment, study, or practice activities. In some embodiments, input from sensor 112 may be correlated with assessment test results and incorporated in a learner attribute profile.

Assessment and behavior inputs logical circuit 110 may communicatively couple to data store 120. Data store 120 may include a database housed on local storage, network attached storage, cloud-based storage, a storage area network, or other data storage devices. Data store 120 may also include historical data regarding the same or other learners' interactions with the systems to empirically determine good and/or bad learning characteristics, habits, and/or behaviors as correlated with successful or unsuccessful learning patterns. For example, a population of learners may exhibit a similar set of learning characteristics, habits, and/or behaviors that correlate to a successful and efficient mastery of a particular skill or attribute. That empirical data may be stored in data store 120 for future correlation to the same or similar learning characteristics, habits, and/or behaviors demonstrable in an individual learner.

Both assessment and behavior input logical circuit 110 and data store 120 may be communicatively coupled to learning analytics server 130. As used herein, communicatively coupled may mean a direct or indirect connection between or among the entities to exchange information (e.g., in the form of electrical or electromagnetic signals) with each other over a wired or wireless communication link. Examples of such a communication link can include a communication bus, a hardwired communication interface (e.g., wire, cable, fiber, and so on), an optical or RF interface, a local area network, a wide area network, a wireless network, or other electronic or electromagnetic communication links.

Learning analytics server 130 may include a computer processor and a non-transitory computer readable medium with computer executable instructions embedded thereon. The computer executable instructions may be configured to perform response feature extraction, machine learning, model training, analytical evaluation of learning performance, and output of feedback to a learner interface in response to one or more inputs. In some examples, the analytical evaluation of learning performance may be implemented according to methods disclosed herein. For example, learning analytics server 130 may determine a learner's level of skill mastery for one or more skills by applying CDM, BKT, behavioral characteristics, and/or socioemotional characteristics to assessment or practice data from a learner, automated examination scoring as disclosed herein. The level of mastery of a particular skill may be stored in the format of a learner skill profile. The learner skill profile may incorporate a Q-matrix, which may include one or more assessment questions and corresponding skills, together with an indication as to whether the learner correctly answered the assessment question and/or a probability that the learner may correctly answer the assessment question in the future based on historical response data to the same or similar assessment questions. The level of mastery may also include a probability of how Learning analytics server 130 may also correlate the foregoing inputs with empirical data from data store 120.

In some embodiments, learning analytics server 130 may include an assessment analytics logical circuit 124 configured to apply a CDM process to an assessment data set to evaluate learner performance. In some embodiments, assessment analytics circuit 124 may provide feedback to a learner interface to improve learner performance. The level of mastery of a given skill may be represented as a probability that the learner will correctly answer a subsequent assessment question selected from the same skill. For example, a skill may be addition. A learner may be presented an assessment with a subset of questions selected from the skill (i.e., addition). The learner's performance on a first group of the subset of questions may be analyzed by assessment analytics logical circuit 124 to determine how many questions the learner is answering correctly, and how challenging each of those questions may be.

Assessment analytics logical circuit 124 may also receive behavioral and/or socioemotional characteristics for the learner, for example, from assessment and behavior inputs logical circuit 110. Such inputs may include the amount of time the learner has spent practicing addition, the learner's psychological profile, how often the learner interacts with others in chat rooms or online discussions on the topic of addition, the learner's general studying habits, and so on. Assessment analytics logical circuit 124 may also receive empirical data demonstrating how fast learners with similar behavioral and socioemotional characteristics may learn the particular skill (e.g., addition). Assessment analytics logical circuit 124 may then analyze some or all of these data inputs to determine a probability that the learner has achieved a threshold level of mastery of the skill (e.g., the learner's probability of answering a question within the skill exceeds a threshold value).

In some embodiments, learning analytics server 130 also includes Bayesian analytics logical circuit 126. Bayesian analytics logical circuit 126 may provide additional input to assessment analytics logical circuit 124 in evaluating a learner's level of mastery of one or more skills. For example, Bayesian analytics logical circuit 126 may apply a Bayesian Knowledge Tracing process to a set of assessment answers received from the learner (e.g., from learner interface 140) to determine the probability that the learner will answer the next question correctly. The Bayesian Knowledge Tracing process is disclosed in more detail below, but generally determines a probability that a question selected from a particular subset of assessment questions corresponding to a particular skill (e.g., addition) will be answered correctly based on a learner's previous performance and rate of learning on other assessment questions selected from the same subset. The probability may change over time as the learner's rate of correctly answering assessment questions selected from the subset increases over time (i.e., because the learner's mastery level increased).

Learner interface 140 may be a computer, a tablet, a mobile device, or other electronic input devices as known in the art. In some examples, reviewer interface 140 may include a graphical user interface configured to display examination responses and enable a reviewer to score the examination responses. In some examples, learner interface 140 may also accept reasons from the reviewer as to why particular scores were assigned to the examination. Those reasons may be relevant to or assist with iterative modification of the set of extracted response features.

Learner interface 140 may also include a diagnostic interface to view an examination response that was scored by the evaluation server along with a predictive model e.g. a learned decision tree indicating how a score was calculated (e.g., which response features were identified and how those features were weighted in deriving the overall score). Learner interface 140 may also include a configuration interface that enables a learner to know his performance and steps for improvements. change settings and parameters used by examination evaluation server 130. For example, a learner may manually add or remove response features, adjust machine learning parameters, enter corpuses or links thereto, or perform other related system tuning.

Learner interface 140 may also include a feedback module. As the learner interacts with the system by taking assessments, practicing, studying, or performing other learning activities, the learning analytics server 130 may generate feedback and insight to help the learner improve his or her level of mastery for one or more skills. In some embodiments, the learner may select or be assigned a learning goal or set of goals (e.g., master addition within the next five days). Learning analytics server 130 may then generate feedback based on the learner's current level of mastery of addition, rate of learning, and related behavioral and socioemotional characteristics, and empirical data received from data store 120. Learning analytics server may then determine that learners with similar behaviors (e.g., study habits), socioemotional characteristics (e.g., psychological profile and tendencies), and assessment performance on assessment questions relating to addition would benefit by implementing a particular study regiment, e.g., by practicing certain questions, receiving tutoring, studying more or less often, getting more sleep, and so on. This feedback may be presented to the learner via learner interface 140, and the learner's particular goals may be updated.

FIG. 1B illustrates an example multidimensional holistic model for dynamic learning diagnostics and feedback. For example, the multidimensional holistic model may include a learning analytics process 150. The learning analytics process 150 may be performed by learning analytics server 130 and may include analyzing multidimensional learning data. The multidimensional learning data may include assessment data 152, which may include data relating to an assessment examination that includes a set of assessment questions. Assessment data 152 may also include characteristics associated with each assessment question, including question difficulty, and a corresponding skill classification. Assessment data 152 may also include a learner's responses to the assessment questions, e.g., as received from a learner interface 140, as well as historical responses to the same assessment questions from the same learner or other learners, as received from data store 120. Analysis of the assessment data may be performed according to methods disclosed herein.

Learning analytics process 150 may also be applied to test preparation data 154. For example, test preparation data 154 may include data indicating the tests preparation activities performed by a learner with respect to one or more skills. In some examples, test preparation data 154 includes a time spent studying one or more skills, data from practice questions, quizzes, and or examinations, performance on skill simulators, time spent and performance on related educational games or simulators, together with other types of test preparation data as known in the art.

Learning analytics process 150 may also be applied to socioemotional data 156. For example, socioemotional data 156 may include a psychological profile for the learner. The psychological profile may be acquired using a questionnaire or other type of interactive answer and response system to deliver and score a psychological evaluation as known in the art. In some examples, the psychological evaluation may be a Myers-Briggs Type Indicator (MBTI), or similar, personality assessment. Socioemotional data 156 may also include data about a learner's current mood, e.g., happy, sad, angry, frustrated, or otherwise. In some embodiments, the mood data may be acquired by the way of self-assessment instruments, or may be signaled by the inferences made on the basis of learner analytics process 150 by the Learning Analytics Server 130 or via externally acquired indicators. For example, the learner may select a particular emoji within the learner interface or a social media application that indicates the learner's current mood. Socioemotional data 156 may also include empirical data regarding the learner's interaction with third-party applications. For example, the learner's propensity to use online forums, chat rooms, social media tools, blogs, articles, e-books, or other applications to interact with other learners, teachers, tutors, or experts in a community, or to access and interact information and tools to assist with learning. Socioemotional data 156 may also include historical benchmark data stored in data store 120 regarding socioemotional data from the same learner, or different learners, as correlated with learner performance metrics, such as performance on assessments.

Learner analytics process 150 may also be applied to behavioral data 158. For example, behavioral data 158 may include empirical data captured from a learner's historical practice and test taking activities. For example, behavioral data 158 may include a learner's study habits, including the learner's propensity to take practice assessments or quizzes, use simulators, or interact with system learning tools without interruption, or at regular intervals. Behavioral data 158 may be captured using data from learner interface 140 that monitors when the learner is using the system and the specific tools with which the learner is interacting. Behavioral data 158 may also be acquired from sensor 112 or from a self-assessment of the learner.

In some embodiments, a multidimensional holistic model for dynamic learning diagnostics and feedback 150 includes application of a holistic learning framework 160. For example, holistic learning framework 160 may include receiving multiple data sets from learning analytics process 150 and using each of the multiple data sets to evaluate a learner's performance and provide feedback. The evaluation of the learner's performance may be performed by a diagnostic model 172 applied by assessment analytics logical circuit 124. Feedback may be generated and delivered to learner interface 140 by a feedback model 174, also as applied by assessment analytics logical circuit 124. For example, the diagnostic model 172 may include evaluation of the learner's performance on an assessment using a CDM process to determine the probability that a learner has mastered a skill (i.e., the probability that a learner will correctly answer an assessment question selected from a subset of assessment questions relating to that skill is above a threshold level), and further refined using a BKT process to determine the probability that a learner will correctly answer a subsequent assessment question based on analysis of the learner's response history to previous assessment questions. In some embodiments, probabilities that a learner has mastered a skill or will correctly answer a subsequent assessment question may be included in a learner's attribute profile, which may be a matrix identifying skills and a learner's proficiency within each skill.

In some examples, the learner attribute profile 1032 (rf. FIG. 1C) may also include assessment questions or types of assessment questions correlated to the particular skill, and CDM or BKT outputs. The learner attribute profile may further include weightings for each entry corresponding to test preparation data 154, socioemotional data 156, and/or behavioral data 158. For example, the learner attribute profile may include the probabilities generated by the CDM and/or BKT processes for each skill and assessment question, and corresponding weighting parameters indicating that the learner was in a happy mood when taking the assessment and when practicing the corresponding skill prior to taking the assessment, that the learner is an extravert and had studied the skill at regular intervals, and that the learner sought tutoring or extra assistance with the skill using particular system resources. The diagnostic model 172 may then determine that the learner did not adequately perform on the subset of assessment questions relating to that skill, and take into account the learner's test preparation practices, behavior, and socioemotional state. The feedback model 174 may then correlate this data identified in the learner attribute profile with available system resources and historical data about effective use of those resources, as correlated with learner attribute profiles that are similar to the learner's, and generate a feedback set that includes recommendations for system resources that the learner may use to improve, improvements to study habits, and other insight for the learner as may be appropriate. The assessment evaluation of the learner's performance and corresponding feedback may then be presented to the learner through learner interface 140.

FIG. 1C illustrates a schematic diagram of an example interactive learner interface. The learner interface 140 may be a graphical user interface, for example, as displayed through a computer display or touchscreen device. Learner interface 140 may include evidence input process 1010. For example, evidence input 1010 may include assessment data 1012, e.g., an administered set of assessment questions and learner responses to those questions. Assessment data 1012 may be obtained by learner interface 140 by administering an digital examinations directly in learner interface 140, or may be received from an external system, such as a digital test administration system, a data file, or an OCR scanner. Evidence input 1010 may also include practice data 1014. Practice data 1014 may indicate the learner's studying patterns, behavioral data, socioemotional data, practice examination results and history, historical data relating to the learner, or to other learners or groups of learners, or other data relating to evaluating learner performance.

Learner interface 140 may also include analytics processes 1020. For example, the analytics processes 1020 may include application of a CDM process 1022 to the assessment data 1012 to generate learner attribute profiles 1032. Learner attribute profiles 1032 may include learner specific matrices identifying assessment questions, corresponding skills, and a probability that the learner has reached a threshold level of mastery of the respective skill, e.g., the probability that the learner will correctly answer an assessment question selected from a subset of assessment questions relating to the skill.

Analytics processes 1020 may also include application of a BKT process 1024 to generate proficiency estimates 1034. Proficiency estimates 1034 may identify, for each learner, a probability that the learner will correctly answer a subsequent assessment question selected from a subset of assessment questions relating to one or more skills based on the learner's previous performance answering similar assessment questions. The BKT process may use as inputs assessment data 1012, practice data 1014, and/or output from CDM process 1022.

Analytics processes 1020 may also include a data capture process 1026 configured to obtain behavioral and socioemotional data 156 and 158. An integrated analytics logical circuit may then evaluate a learner's performance and current level of mastery for one or more skills as a function of the learner attribute profiles 1032, proficiency estimates 1034, socioemotional data 156, and/or behavioral data 158. In some examples, the integrated analytics logical circuit may correlate with the learner attribute profile 1032 to one or more of a proficiency estimate 1034, socioemotional data set 156, and behavioral data set 158, with respect to one or more skills and related subsets of assessment questions.

Analytics processes 1020 may also include a feedback process. Within the feedback process, a resource recommendation process may obtain a list of resources 1038, e.g., videos 1042, quizzes 1044, games 1046, or simulations 1048, and an output from integrated analytics logical circuit 1030. The resource recommendations process 1038 may also obtain historical data from a data store and analyze, based on the learner's performance, historical data, and available learning resources, a recommended learning plan for the particular learner to achieve a particular goal (e.g., improving on one or more skills). The feedback process may also include an insight process 1039 to give the learner more general study and examination taking tips, including strategies of addressing learner-specific behavioral or socioemotional deficiencies.

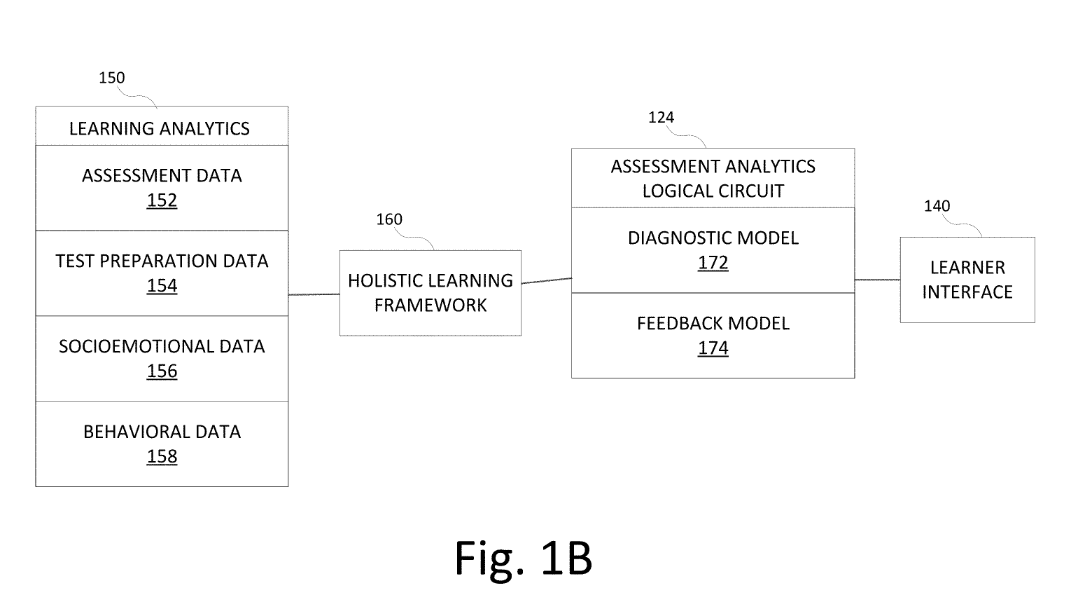

FIG. 2 is a flow chart illustrating an example method for dynamic assessment and feedback. For example, the method for dynamic assessment and feedback 200 may include presenting a set of assessment questions at step 205 and obtaining a set of corresponding responses to the assessment questions at step 210. The set of assessment questions may elicit a finite answer, such as true or false, or multiple choice. In some embodiments, the set of assessment questions may elicit a numerical answer, a single character or one-word answer, a short answer, or an essay. The set of assessment questions may be presented through a learner interface, via paper examination or quiz, orally, or via other methods of administering an examination as known in the art. Likewise, a learner's responses to the assessment questions may be obtained via a learner interface, by scanning a paper response sheet, or entered via other examination response acquisition methods as known in the art.

Method 200 may further include obtaining diagnostic scoring rules and parameters at step 215 and applying the diagnostic scoring rules to responses to generate a learner response matrix at step 225. For example, the scoring rules and parameters may include information to determine whether answers obtained in response to the set of assessment questions are correct. For example, the scoring rules may include a scoring key, or automated scoring algorithm. In some examples, the scoring rules and parameters may include a level of difficulty for each assessment question and a categorization of the assessment question into one or more skills. Skills may include granular or broad categorizations of skills, for example, math, addition, subtraction, multiplication, division, linear algebra, calculus, differential equations, finite math, and geometry may each be a skill. These skills may include more granular sub-skills. The diagnostic scoring rules and parameters may then be applied to a set of learner responses to the set of assessment questions to determine which questions the learner answered correctly, and corresponding information about those questions, including the respective skills and level of difficulty. One or more assessment questions may then be populated into a learner response matrix together with an indication as to whether the learner correctly answered the question, the question's level of difficulty, and the skills corresponding to the question.

In some embodiments, method 200 may also include obtaining learner-specific behavioral and/or socioemotional parameters at step 220. These parameters may include socioemotional data 156 and/or behavioral data 158 and may be populated, together with one or more learner response matrices, in a learner attribute profile at step 230. Generating a learner attribute profile at step 230 may further include generating a probability that a learner has mastered one or more skills. For example, the method may determine the probability that a learner has mastered a skill by evaluating the number of correct assessment responses to questions relating to that skill, and weighting each correct response with a difficult of the respective assessment question. The probability that a learner has mastered a skill may further be weighted using socioemotional data 156 and/or behavioral data 158. For example, the learner may be depressed, stressed, or upset when taking the assessment, and may have a personality profile in which the learner's performance is dramatically inhibited based on one or more of these negative moods. The method may increase add weight to correct responses and reduce weight from incorrect responses under these particular circumstances.

In some examples, method 200 may further include estimating a learner response to a subsequent assessment question by applying a Bayesian knowledge tracing (BKT) process to the learner attribute profile at step 235. For example, the BKT process may include evaluation of a learner's previous responses to questions selected from a subset of assessment questions relating to one or more skills to determine the probability that the learner will response correctly to the next question presented from the same subset of assessment questions.

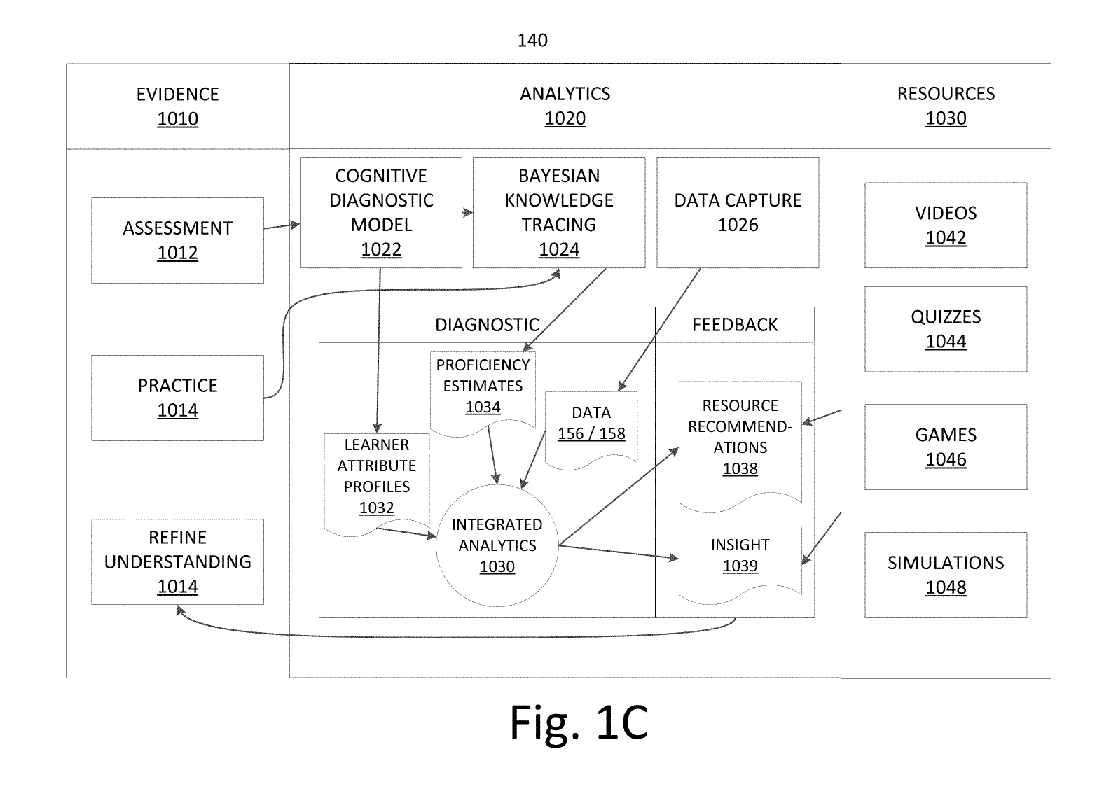

FIG. 3 is a flow chart illustrating an example BKT process. Referring to FIG. 3, a BKT process 300 may include presenting a subset of assessment questions from a common category (e.g., from a subset of assessment questions relating to one or more skills) at step 305 and determining a category-specific learning rate and updated learner attribute profile by tracing the accuracy of the responses to those assessment questions over time at step 310. BKT process may be implemented to as a two-state learning model to determine whether a particular skill is either learned or unlearned. The learner attribute profile may then be updated with the output from the BKT process for each skill. The update to the skill masteries may be determined by tracing indications as to whether a learner's response to one or more assessment questions is correct over a given time period. Both of these models are described in more detail below.

BKT process 300 may also include predicting a learner's anticipated response to a subsequent question within the common category as a function of the current skill mastery as stored in the learner attribute profile at step 315. For example, the method may include anticipating whether a learner will answer a given question correctly or incorrectly based on the probabilistic output from the BKT process. In some examples, the prediction in step 315 may be weighted based on the difficulty level of the question presented, socioemotional data 156, and/or behavioral data 158. BKT process 300 may also include generating a multi-state Bayesian knowledge matrix for the common category at step 320. This matrix may include the learner attribute profile together with the Bayesian probability outputs and learning rates to provide a complete learning profile for the learner, representing the current learning state of the learner with respect to one or more skills.

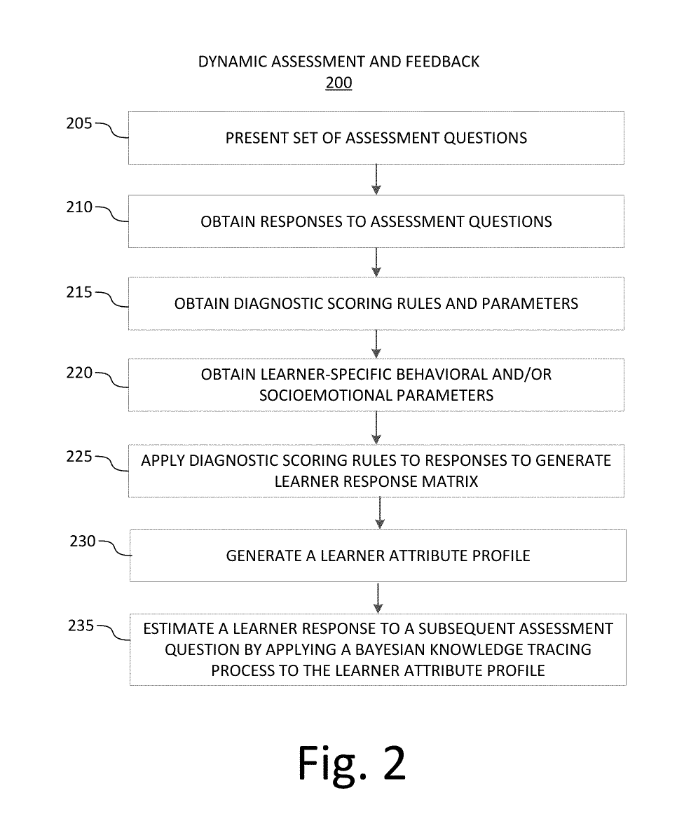

FIG. 4 is a flow chart illustrating an example learner analytics process. Learning analytics process 400 may include determining if one or more learner-specific behavioral parameters correlate to at-risk behavior. For example, the learner-specific behavioral parameters may be selected from socioemotional data 156 and/or behavioral data 158, and may indicate a learner's propensity to at-risk behavior, learning needs, attention span, ability to focus and absorb information, or other behavioral and/or socioemotional learning characteristics. The determination as to whether these behavioral characteristics correlate to at risk behaviors may be determined based on manual review and user input, or from empirical study and data mining of previously acquired data sets.

Method 400 may also include correlating learner-specific behavioral parameters to the learning rate and/or learner attribute profile at step 410. The determination as to whether a behavioral parameter correlates to an at-risk behavior from step 405 may then include filtering the empirical data mined from previously acquired data sets by their respective learner attribute profiles as compared with the current learner's attribute profile. The method may further include presenting a set of behavioral improvement recommendations at step 415.

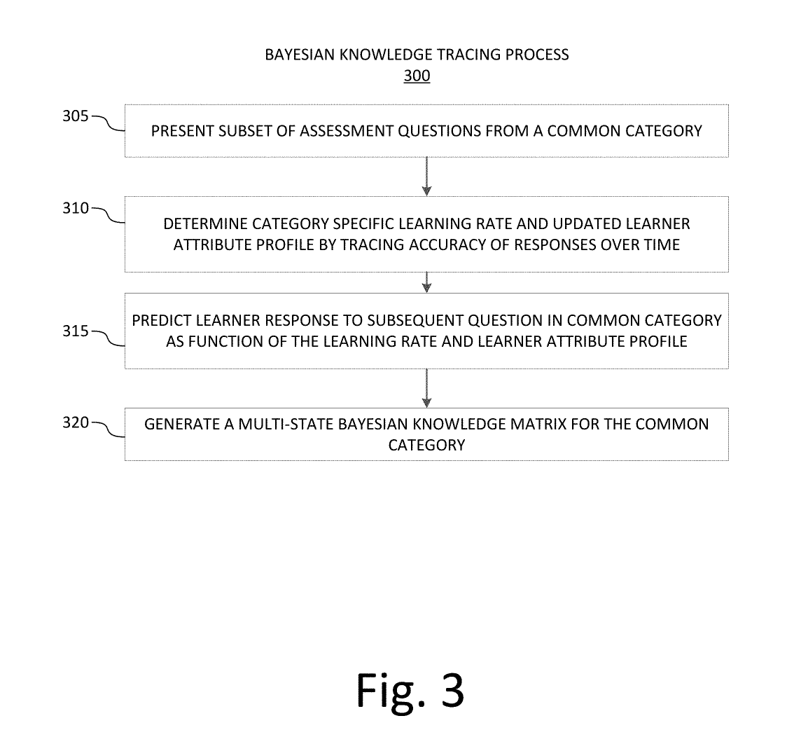

FIG. 5 illustrates an example Q-matrix as used in connection with embodiments disclosed herein. In the example illustrated by FIG. 5, the first question corresponds to a skill of addition, the second attribute corresponds to a skill of subtraction, the third question corresponds to a skill of division, and the fourth question corresponds to skills of subtraction and multiplication. Notably, each question corresponds to a skill of math. Other example Q-matrices for other skills may be used across any type of skill category. The Q-matrix data may then be included in the learner attribute profile together with a probability that the learner will answer a question within that skill correctly based on previous responses.

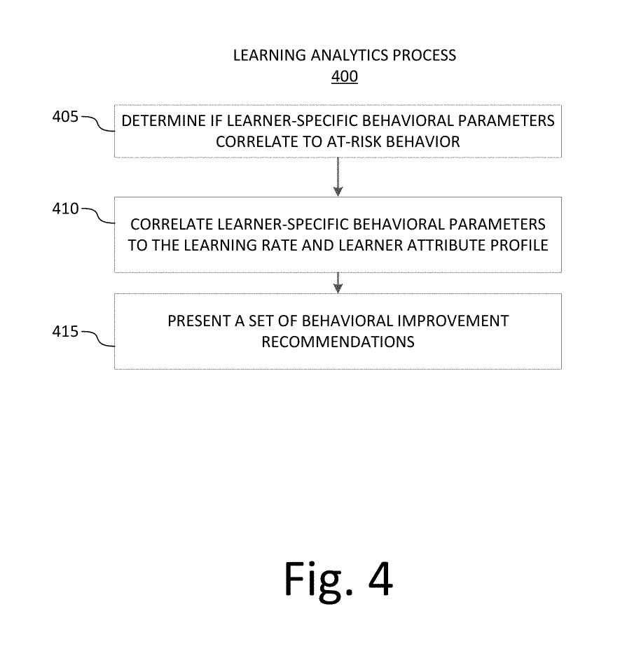

FIG. 6 is an example representation of an output from a CDM analysis as used in connection with embodiments disclosed herein. For example, the chart illustrated in FIG. 6 corresponds to the Q-matrix in FIG. 5 and corresponding response correctness indications from a learner. The probabilities may be weighted positively or negatively based on the relative difficulty of each question, as well as socioemotional data 156 and/or behavioral data 158. In the example illustrated, the learner has sufficiently correctly answered questions within the addition skill matrix to generate a probability of 82% of correctly answering a question within that skill. The learner has a 75% probability of answering a subtraction question correctly, but only a 33% probability of answering a multiplication question correctly and a 25% chance of answering a division question correctly. In the example, the mastery threshold is set at 50%, such that the learner has mastered addition and subtraction, but not multiplication or division. A higher or lower mastery threshold may be set. For this example, the learner attribute profile may be updated with the probability and/or binary indication of skill mastery for each skill. Examples with respect to other skills and Q-matrices may be used. The mastery threshold may be set higher or lower based on user preferences and tuning. For example, the mastery threshold may be about or greater than 75%. In some examples, the mastery threshold may be about or greater than 95%.

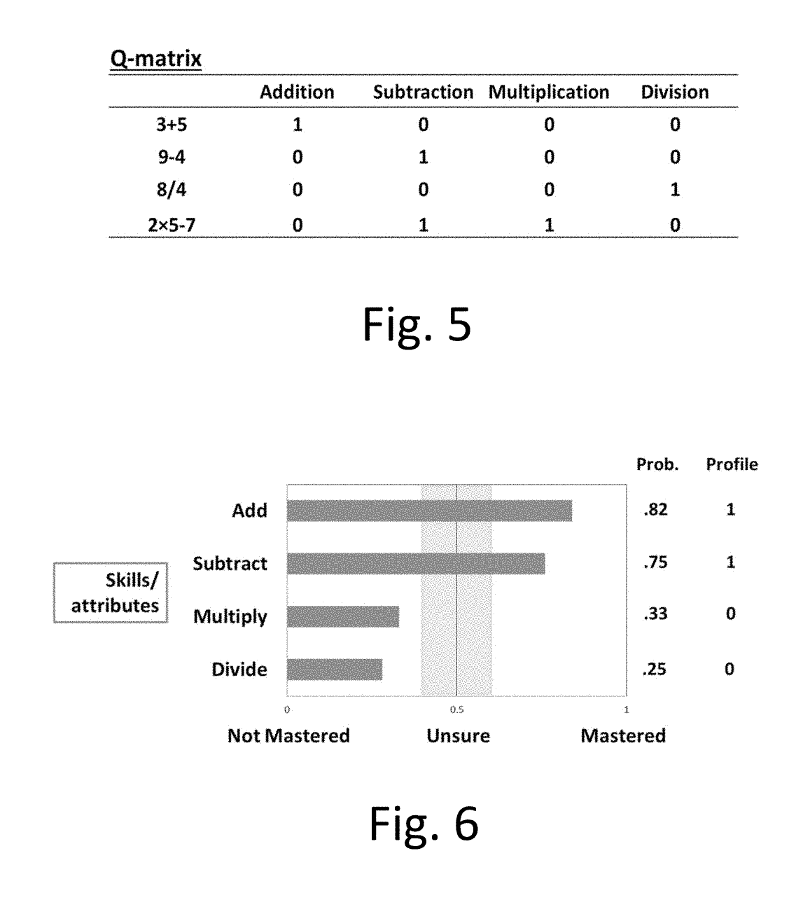

FIG. 7 is a diagram illustrating an example of a BKT process. For example, responses may be tracked over time for each question selected within the common category to determine a correct response rate. For a particular skill, a learner's response sequence 1 to n may be used to predict the response to question n+1. The prediction may be based on a sequence of items that are dichotomously scored, each item corresponding to a skill. The BKT process may track the learner's knowledge over time based on the learner's performance. The learner may learn on each question, for example, with the assistance of learning resources and feedback. Thus, the correct response rate may change over time.

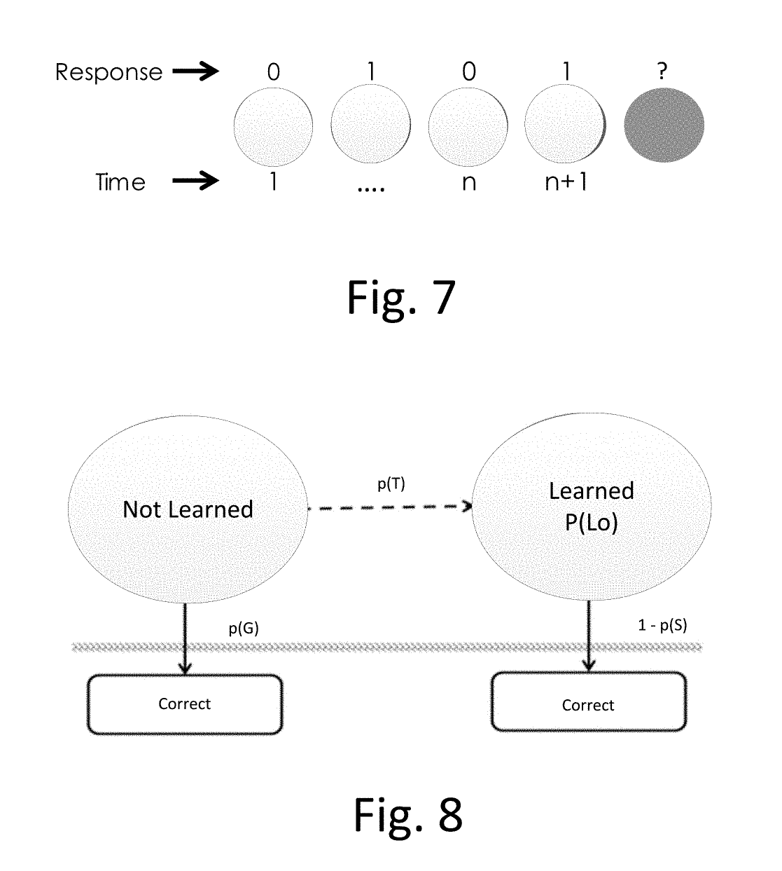

FIG. 8 is a diagram illustrating an example BKT tracing process represented as a hidden Markov model. This is a two-state learning model in which a skill may be either learned or unlearned. The example illustrated in FIG. 8 includes four parameters. The parameter p(L₀) represents the probability that the skill is already known before the first opportunity to use the skill in problem solving. The parameter p(T) represents the probability that the skill will be learned at each opportunity to use the skill, regardless of whether the answer is correct or incorrect. The parameter p(G) represents the probability that the learner will guess correctly if the skill is not known. The parameter p(S) represents the probability that the learner will incorrectly answer a question even if the skill is learned. Using this model, a BKT process may generate a probability that the skill is learned.

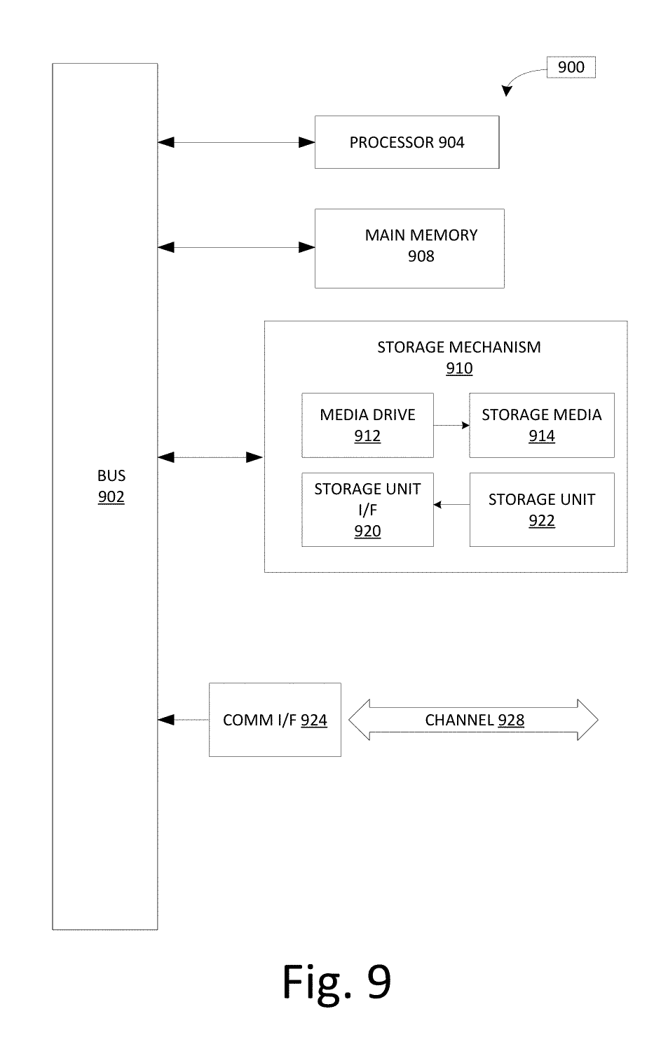

As used herein, the terms logical circuit and engine might describe a given unit of functionality that can be performed in accordance with one or more embodiments of the technology disclosed herein. As used herein, either a logical circuit or an engine might be implemented utilizing any form of hardware, software, or a combination thereof. For example, one or more processors, controllers, ASICs, PLAs, PALs, CPLDs, FPGAs, logical components, software routines or other mechanisms might be implemented to make up a engine. In implementation, the various engines described herein might be implemented as discrete engines or the functions and features described can be shared in part or in total among one or more engines. In other words, as would be apparent to one of ordinary skill in the art after reading this description, the various features and functionality described herein may be implemented in any given application and can be implemented in one or more separate or shared engines in various combinations and permutations. Even though various features or elements of functionality may be individually described or claimed as separate engines, one of ordinary skill in the art will understand that these features and functionality can be shared among one or more common software and hardware elements, and such description shall not require or imply that separate hardware or software components are used to implement such features or functionality.

Where components, logical circuits, or engines of the technology are implemented in whole or in part using software, in one embodiment, these software elements can be implemented to operate with a computing or logical circuit capable of carrying out the functionality described with respect thereto. One such example logical circuit is shown in FIG. 9. Various embodiments are described in terms of this example logical circuit 900. After reading this description, it will become apparent to a person skilled in the relevant art how to implement the technology using other logical circuits or architectures.

Referring now to FIG. 9, computing system 900 may represent, for example, computing or processing capabilities found within desktop, laptop and notebook computers; hand-held computing devices (PDA's, smart phones, cell phones, palmtops, etc.); mainframes, supercomputers, workstations or servers; or any other type of special-purpose or general-purpose computing devices as may be desirable or appropriate for a given application or environment. Logical circuit 900 might also represent computing capabilities embedded within or otherwise available to a given device. For example, a logical circuit might be found in other electronic devices such as, for example, digital cameras, navigation systems, cellular telephones, portable computing devices, modems, routers, WAPs, terminals and other electronic devices that might include some form of processing capability.

Computing system 900 might include, for example, one or more processors, controllers, control engines, or other processing devices, such as a processor 904. Processor 904 might be implemented using a general-purpose or special-purpose processing engine such as, for example, a microprocessor, controller, or other control logic. In the illustrated example, processor 904 is connected to a bus 902, although any communication medium can be used to facilitate interaction with other components of logical circuit 900 or to communicate externally.

Computing system 900 might also include one or more memory engines, simply referred to herein as main memory 908. For example, preferably random access memory (RAM) or other dynamic memory, might be used for storing information and instructions to be executed by processor 904. Main memory 908 might also be used for storing temporary variables or other intermediate information during execution of instructions to be executed by processor 904. Logical circuit 900 might likewise include a read only memory (“ROM”) or other static storage device coupled to bus 902 for storing static information and instructions for processor 904.

The computing system 900 might also include one or more various forms of information storage mechanism 910, which might include, for example, a media drive 912 and a storage unit interface 920. The media drive 912 might include a drive or other mechanism to support fixed or removable storage media 914. For example, a hard disk drive, a floppy disk drive, a magnetic tape drive, an optical disk drive, a CD or DVD drive (R or RW), or other removable or fixed media drive might be provided. Accordingly, storage media 914 might include, for example, a hard disk, a floppy disk, magnetic tape, cartridge, optical disk, a CD or DVD, or other fixed or removable medium that is read by, written to or accessed by media drive 912. As these examples illustrate, the storage media 914 can include a computer usable storage medium having stored therein computer software or data.

In alternative embodiments, information storage mechanism 190 might include other similar instrumentalities for allowing computer programs or other instructions or data to be loaded into logical circuit 900. Such instrumentalities might include, for example, a fixed or removable storage unit 922 and an interface 920. Examples of such storage units 922 and interfaces 920 can include a program cartridge and cartridge interface, a removable memory (for example, a flash memory or other removable memory engine) and memory slot, a PCMCIA slot and card, and other fixed or removable storage units 922 and interfaces 920 that allow software and data to be transferred from the storage unit 922 to logical circuit 900.

Logical circuit 900 might also include a communications interface 924. Communications interface 924 might be used to allow software and data to be transferred between logical circuit 900 and external devices. Examples of communications interface 924 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 924 might typically be carried on signals, which can be electronic, electromagnetic (which includes optical) or other signals capable of being exchanged by a given communications interface 924. These signals might be provided to communications interface 924 via a channel 928. This channel 928 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 908, storage unit 920, media 914, and channel 928. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the logical circuit 900 to perform features or functions of the disclosed technology as discussed herein.

Although FIG. 9 depicts a computer network, it is understood that the disclosure is not limited to operation with a computer network, but rather, the disclosure may be practiced in any suitable electronic device. Accordingly, the computer network depicted in FIG. 9 is for illustrative purposes only and thus is not meant to limit the disclosure in any respect.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent engine names other than those depicted herein can be applied to the various partitions.

Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “engine” does not imply that the components or functionality described or claimed as part of the engine are all configured in a common package. Indeed, any or all of the various components of an engine, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

I claim:

1. A computer implemented method of dynamically assessing and providing feedback to a learner, the method comprising:
displaying, on a learner interface, a set of assessment questions;
obtaining, from the learner interface, a set of responses corresponding to the assessment questions;
obtaining a set of diagnostic scoring rules, each scoring rule comprising a set of diagnostic parameters corresponding to each assessment question and a response key;
obtaining a set of learner-specific behavioral parameters;
applying the set of diagnostic scoring rules to the set of responses to generate a learner response matrix;
generating a learner attribute profile by applying as a set of probabilities of mastering each learning category to the learner response matrix; and
estimating a learner response to a subsequent assessment question by applying a cognitive diagnostic model (CDM) or a Bayesian knowledge tracing (BKT) process to the learner attribute profile.

2. The computer implemented method of claim 1 wherein the learner attribute profile further comprises the set of learner-specific behavioral parameters.

3. The computer implemented method of claim 1, wherein the learner response matrix comprises a list of categories and a level of skill accrued by the learner with respect to each category.

4. The computer implemented method of claim 2, wherein the BKT process comprises:
displaying, with the learner interface, a subset of assessment questions wherein each question in the subset is selected from a common category;
determining a skill-specific mastery value and updated learner attribute profile by tracing an accuracy of each sequential response to each question of the subset of assessment questions; and
predicting a learner response to a subsequent assessment question from the subset of assessment questions as a function of the skill-specific mastery value and the updated learner attribute profile.

5. The computer implemented method of claim 4, wherein the BKT process further comprises generating a multi-state Bayesian knowledge vector corresponding to the common category, the multi-state Bayesian knowledge vector comprising a first parameter indicating whether the category is presently mastered and a second parameter indicating the probability that the category will be mastered within a threshold timeframe as a function the skill-specific mastery value and updated learner attribute profile.

6. The computer implemented method of claim 4, further comprising determining if any of the learner-specific behavioral parameters correlate to at-risk behavior.

7. The computer implemented method of claim 5, further comprising correlating the set of learner-specific behavioral parameters to the learning rate and the learner attribute profile.

8. The computer implemented method of claim 5, further comprising presenting, to the learner-interface, a set of behavioral improvement recommendations to correct at-risk behavior.

9. The computer implemented method of claim 5, further comprising presenting, to the learner interface, a set of behavioral improvement recommendations to increase a learning rate.

10. The computer implemented method of claim 5, further comprising presenting, to the learner interface, a set of behavioral improvement recommendations to increase the skill-specific mastery value.

11. The computer implemented method of claim 1, wherein the learner-specific behavioral parameters comprise a type of learning resource accessed by the learner, a time spent by the learner on a task, a participation level of the learner with an interactive interface, or a persistence ratio of a number of times retaking an assessment compared with the probability that one or more skills from the category will be mastered.

12. The computer implemented method of claim 1, wherein obtaining the learner-specific behavioral parameters comprises receiving behavioral indications from a learner input device.

13. The computer implemented method of claim 12, wherein the learner input device comprises a mouse, a microphone, a keyboard, or a touchscreen.

14. The computer implemented method of claim 4, wherein determining if any of the learner-specific behavioral parameters correlate to at-risk behavior comprises obtaining historical behavioral data from a historical assessment database.

15. A system for dynamically assessing and providing feedback to a learner, the system comprising:
a learner interface, a data store, and an assessment analytics logical circuit;
wherein the assessment analytics logical circuit comprises a processor and a non-transitory medium with computer executable instructions embedded thereon, the computer executable instructions to cause the processor to:
display a set of assessment questions on the learner interface;
obtain, from the learner interface, a set of responses corresponding to the assessment questions;
obtain a set of diagnostic scoring rules, each scoring rule comprising a set of diagnostic parameters corresponding to each assessment question and a response key;
obtain a set of learner-specific behavioral parameters;
apply the set of diagnostic scoring rules to the set of responses to generate a learner response matrix;
generate a learner attribute profile by applying as a set of probabilities of mastering each learning category to the learner response matrix; and
estimate a learner response to a subsequent assessment question by applying a CDM or a Bayesian knowledge tracing (BKT) process to the learner attribute profile.

16. The system of claim 15 wherein the learner attribute profile further comprises the set of learner-specific behavioral parameters.

17. The system of claim 15, wherein the learner response matrix comprises a list of categories and a level of skill accrued by the learner with respect to each category.

18. The system of claim 16, wherein the computer executable instructions further cause the processor to:
display a subset of assessment questions on the learner interface, wherein each question in the subset is selected from a common category;
determine a skill-specific mastery value and updated learner attribute profile by tracing an accuracy of each sequential response to each question of the subset of assessment questions; and
predict a learner response to a subsequent assessment question from the subset of assessment questions as a function of the skill-specific mastery value and the updated learner attribute profile.

19. The system of claim 18, wherein the computer executable instructions further cause the processor to generate a multi-state Bayesian knowledge vector corresponding to the common category, the multi-state Bayesian knowledge vector comprising a first parameter indicating whether the category is presently mastered and a second parameter indicating the probability that the category will be mastered within a threshold timeframe as a function the skill-specific mastery value and updated learner attribute profile.

20. The system of claim 18, wherein the computer executable instructions further cause the processor to determine if any of the learner-specific behavioral parameters correlate to at-risk behavior.

21. The system of claim 20, wherein the computer executable instructions further cause the processor to correlate the set of learner-specific behavioral parameters to the learning rate and the learner attribute profile.

22. The system of claim 20, wherein the computer executable instructions further cause the processor to present a set of behavioral improvement recommendations to the learner-interface to correct at-risk behavior.

23. The system of claim 20, wherein the computer executable instructions further cause the processor to present a set of behavioral improvement recommendations, on the learner interface to increase a learning rate.

24. The system of claim 20, wherein the computer executable instructions further cause the processor to present a set of behavioral improvement recommendations on the learner interface to increase the skill-specific mastery value.

25. The system claim 15, wherein the learner-specific behavioral parameters comprise a type of learning resource accessed by the learner, a time spent by the learner on a task, a participation level of the learner with an interactive interface, or a persistence ratio of a number of times retaking an assessment compared with the probability that one or more skills from the category will be mastered.

26. The system of claim 15, wherein the computer executable instructions further cause the processor to receive behavioral indications from a learner input device.

27. The system of claim 26, wherein the learner input device comprises a mouse, a microphone, a keyboard, or a touchscreen.

28. The system of claim 18, wherein the computer executable instructions cause the processor to determine if any of the learner-specific behavioral parameters correlate to at-risk behavior by obtaining historical behavioral data from a historical assessment database.
